(19) United States
(12) Patent Application Publication
(10) Pub. No.: US 2009/0318808 A1
(43) Pub. Date: Dec. 24, 2009

(54) ULTRASOUND DEVICE AND SYSTEM INCLUDING SAME

(76) Inventor: Eric William BRADER, Wexford, PA (US)

Correspondence Address: REED SMITH LLP, P.O. BOX 488, PITTSBURGH, PA, 15230-0488, US

(21) Appl. No.: 12/467,922

(22) Filed: May 18, 2009

Related U.S. Application Data
(60) Provisional application No. 61/053,877, filed on May 16, 2008.

(51) Int. Cl. A61B 8/14

(52) U.S. Cl. 600/443

(57) ABSTRACT

An ultrasound device. The ultrasound device is portable and includes a shock and vibration resistant housing, an ultrasound module positioned within the housing, a processor positioned within the housing, and a display communicably connected to the processor. The ultrasound module is configured for transmitting control signals to a transducer, and for digitizing echo signals received from the transducer. The processor is communicably connected to the ultrasound module, and is configured to generate an image based on the digitized echo signals.

### CROSS-REFERENCE TO RELATED APPLICATION

This application claims the benefit under 35 U.S.C. § 119(e) of the earlier filing date of United States Provisional Patent Application No. 61/053,877 filed on May 16, 2008.

### BACKGROUND

This application discloses an invention which is related, generally and in various embodiments, to an ultrasound device and to a system which includes the ultrasound device.

### SUMMARY

In one general respect, this application discloses a portable ultrasound device. According to various embodiments, the portable ultrasound device includes a shock and vibration resistant housing, an ultrasound module positioned within the housing, a processor positioned within the housing, and a display communicably connected to the processor. The ultrasound module is configured for transmitting control signals to a transducer, and for digitizing echo signals received from the transducer. The processor is communicably connected to the ultrasound module, and is configured to generate an image based on the digitized echo signals.

In another general respect, this application discloses a portable device. According to various embodiments, the portable device includes an ultrasound module, a processor communicably connected to the ultrasound module, a display communicably connected to the processor, and a heart monitor module and/or a defibrillator module communicably connected to the processor. The ultrasound module is configured for transmitting control signals to a transducer, and for digitizing echo signals received from the transducer. The processor is configured to generate an image based on the digitized echo signals.

In yet another general respect, this application discloses a system. According to various embodiments, the system includes a device configured to digitize a signal received from a transducer, and a server communicably connected to the device. The server is configured to generate an image based on the digitized signal.

Aspects of the invention may be implemented by a computing device and/or a computer program stored on a computer-readable medium. The computer-readable medium may comprise a disk, a device, and/or a propagated signal.

### BRIEF DESCRIPTION OF THE DRAWINGS

Various embodiments of the invention are described herein in by way of example in conjunction with the following figures, wherein like reference characters designate the same or similar elements.

FIG. 1 is a high-level representation of an ultrasound device according to various embodiments;

FIG. 2 illustrates various embodiments of the ultrasound device of FIG. 1;

FIG. 3 illustrates a high level representation of an ultrasound device according to various embodiments;

FIG. 4 illustrates a high level representation of an ultrasound device according to various embodiments;

FIG. 5 illustrates various embodiments of a user interface of the ultrasound device of FIG. 1;

FIG. 6 illustrates a high level representation of an ultrasound device according to various embodiments;

FIG. 7 illustrates various embodiments of a system;

FIG. 8 illustrates various embodiments of a transducer;

FIG. 9 illustrates a high level representation of an ultrasound system according to various embodiments; and

FIG. 10 illustrates a positioning of a transmitting probe and an image generating probe of the ultrasound system of FIG. 9.

### DETAILED DESCRIPTION

It is to be understood that at least some of the figures and descriptions of the invention have been simplified to illustrate elements that are relevant for a clear understanding of the invention, while eliminating, for purposes of clarity, other elements that those of ordinary skill in the art will appreciate may also comprise a portion of the invention. However, because such elements are well known in the art, and because they do not facilitate a better understanding of the invention, a description of such elements is not provided herein.

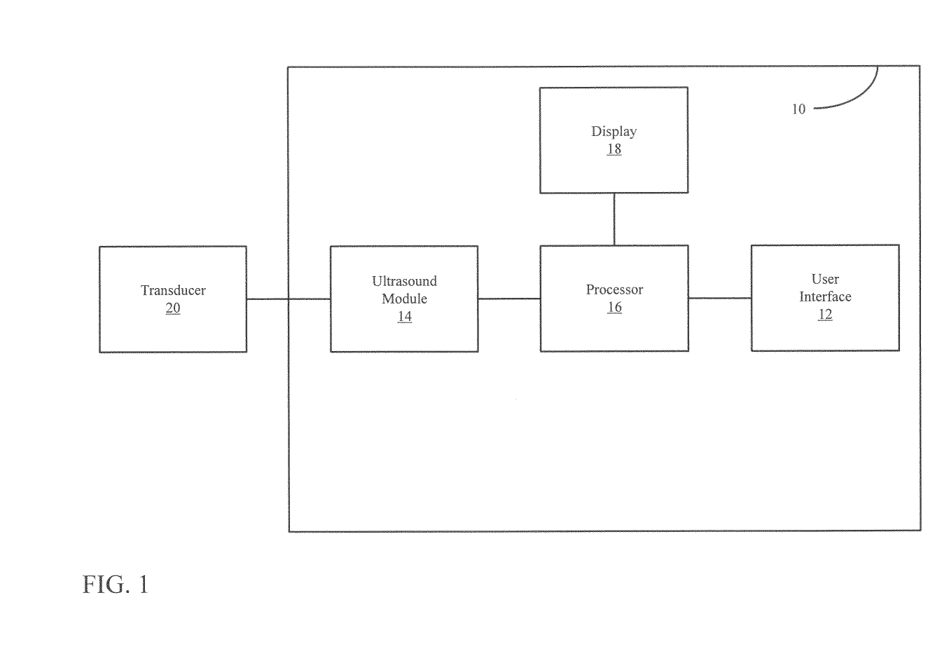

FIG. 1 is a high-level representation of an ultrasound device 10 according to various embodiments. The ultrasound device 10 includes a user interface 12, an ultrasound module 14, a processor 16 communicably connected to the user interface 12 and the ultrasound module 14, and a display 18 communicably connected to the processor 14. The user interface 12 allows a user to control various parameters (e.g., depth, gain, etc.) associated with an ultrasound application. According to various embodiments, the user interface 12 may be embodied as a keyboard having a plurality of input keys, as a touch screen on the display 18, and/or combinations thereof. As shown in FIG. 1, a transducer 20 may be communicably connected to the ultrasound device 10. As described in more detail hereinafter, various embodiments of the ultrasound device 10 may be utilized in medical helicopter applications, in ambulatory unit applications, and in primary care applications.

The ultrasound module 14 is configured to transmit control signals to the transducer 20, to receive echo signals from the transducer 20, and to digitize the received echo signals. The processor 16 is configured to receive the digitized echo signals and to generate images based on the digitized echo signals. According to various embodiments, the ultrasound module 14 is embodied as a chip set similar to those currently offered by Terason Ultrasound, a division of Teratech Corporation of Burlington, Mass.

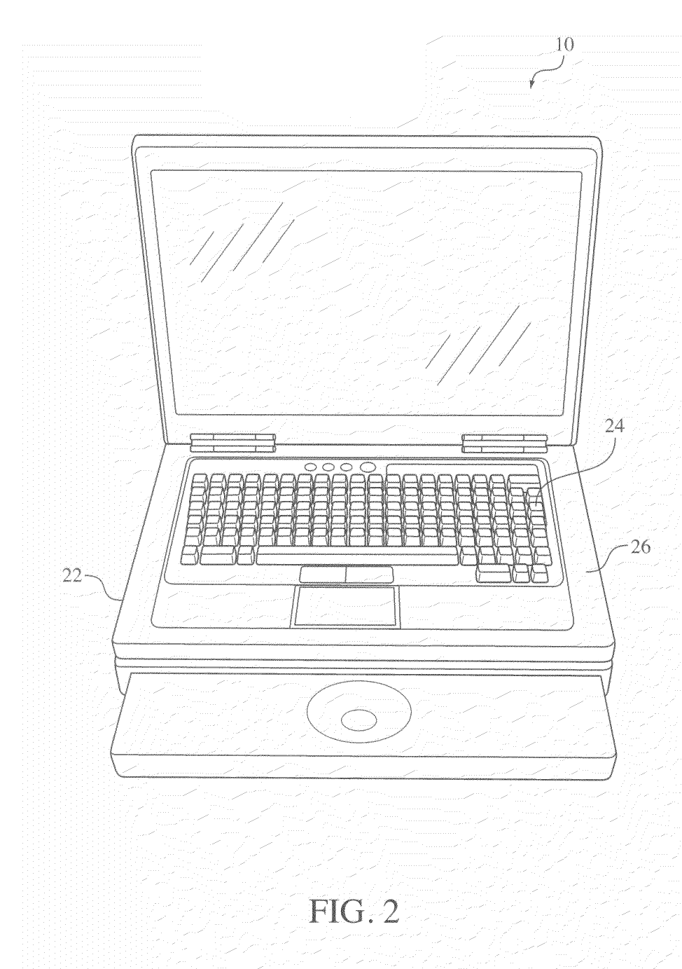

FIG. 2 illustrates various embodiments of the ultrasound device 10 of FIG. 1. The ultrasound device 10 is a portable device, and has a size, shape and weight similar to many of the currently available laptop computers. As shown in FIG. 2, the ultrasound device 10 also includes a housing 22, a plurality of alpha-numeric keys 24, and a port 26 which operates as an interface between the transducer 20 and the ultrasound module 14. The housing 22 houses the ultrasound module 14 and the processor 16. The housing 22 is fabricated from a material having a suitable hardness such that the ultrasound device 10 is able to function properly while being subjected to vibrations, dust, grime, after being dropping onto the ground, etc. For example, according to various embodiments, the housing 22 is fabricated from magnesium, and the device 10 is fabricated in accordance with military standard MIL-STD-810F relating to the ability to withstand drops, shocks, altitude, vibration, etc. The port 26 may be embodied as any suitable type of port. For example, according to various embodiments, the port 26 may be embodied as IEEE 1394 port, a USB port, etc.

Due to its portability and hardness, the ultrasound device 10 may be utilized for medical helicopter applications, ambulatory applications, etc. For example, the ultrasound device 10 may be removably connected to a stationary surface on the interior of a helicopter (e.g., via a bracket) so that the ultrasound device 10 is stationary while the helicopter is in use. Once the helicopter reaches a destination, the ultrasound device 10 can be removed from the interior of the helicopter and taken to a patient in the field.

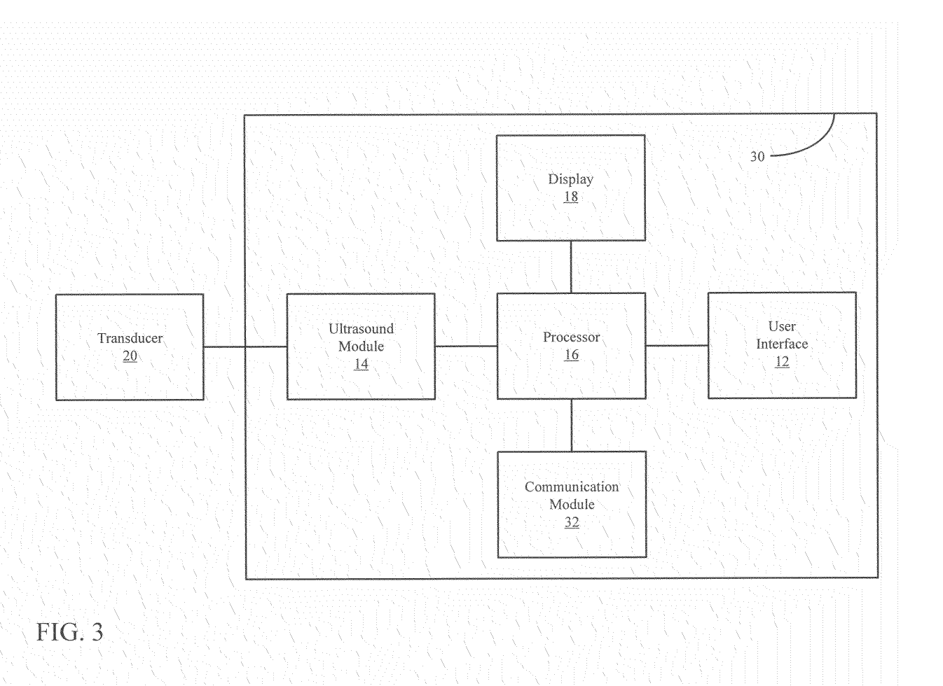

FIG. 3 illustrates a high level representation of an ultrasound device 30 according to various embodiments. The ultrasound device 30 of FIG. 3 is similar to the ultrasound device 10 of FIG. 1, but is different in that it also includes a communication module 32 communicably connected to the processor 16. The communication module 32 is configured to wirelessly transmit information (e.g., the ultrasound images of a patient) from the ultrasound device 10 to a remote computing system (e.g., a hospital computing system) prior to and/or while the patient is being transported. Thus, real-time information regarding the patient will be available, for example, to hospital personnel prior to the time that the patient arrives at the hospital.

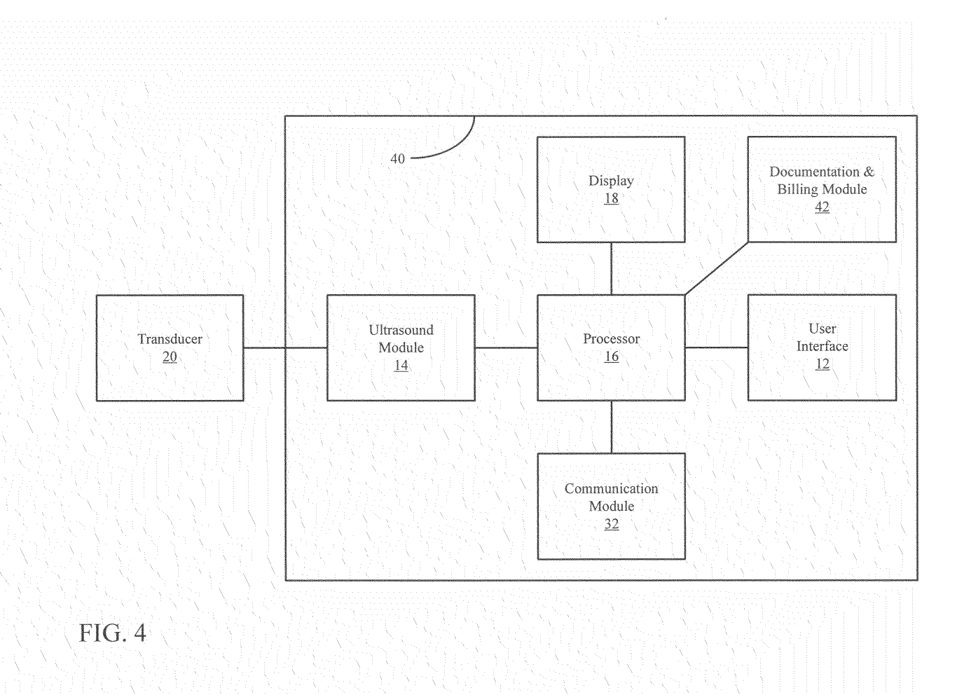

FIG. 4 illustrates a high level representation of an ultrasound device 40 according to various embodiments. The ultrasound device 40 of FIG. 4 is similar to the ultrasound device 30 of FIG. 3, but is different in that it further includes a documentation and billing module 42 communicably connected to the processor 16. The documentation and billing module 42 is configured to attach or append patient information (e.g., patient name, insurance information, date and time the scan was taken, etc.) to a given image generated by the ultrasound device 40. The attached or appended information may be wirelessly communicated to a remote computing system via the communication module 32.

According to other embodiments, the ultrasound device 40 may be utilized in a primary care physician's office, and the information associated with the documentation and billing module 42 may be sent to a computer system at the primary care physician's office via a hardwired connection. Similarly, the ultrasound device 40 may be utilized in an emergency room of a hospital, and the information associated with the documentation and billing module 42 may be sent to a computer system at the physician's office via a hardwired connection. In either case, the sending of the information associated with the documentation and billing module 42 facilitates the billing process and operates to reduce billing errors.

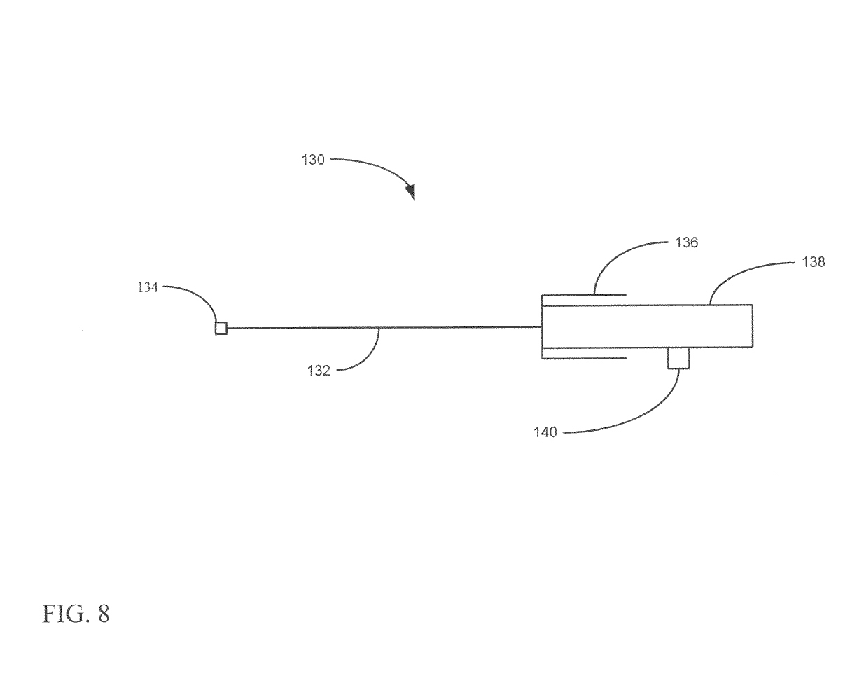

According to various embodiments, the documentation and billing module 42 is communicably connected to the processor 16 via the transducer 20. For such embodiments, the doumentation and billing module 42 is incorporated into a memory device (e.g., a thumb drive) which is removably connected to the probe end of the transducer 20 via, for example, a universal serial bus port in tandem with the transducer cable. With such an arrangement, the transducer 20 may be utilized as a pocket-sized personal transducer that a user may carry from one ultrasound device to another ultrasound device. In such instances, the user may automatically identify himself by logging onto an ultrasound system, may record studies to his own portable drive as well as automatically capturing billing demographics of patients who are already registered with the system, etc. A more detailed description of such a pocket-sized personal transducer is provided hereinbelow with respect to FIG. 8.

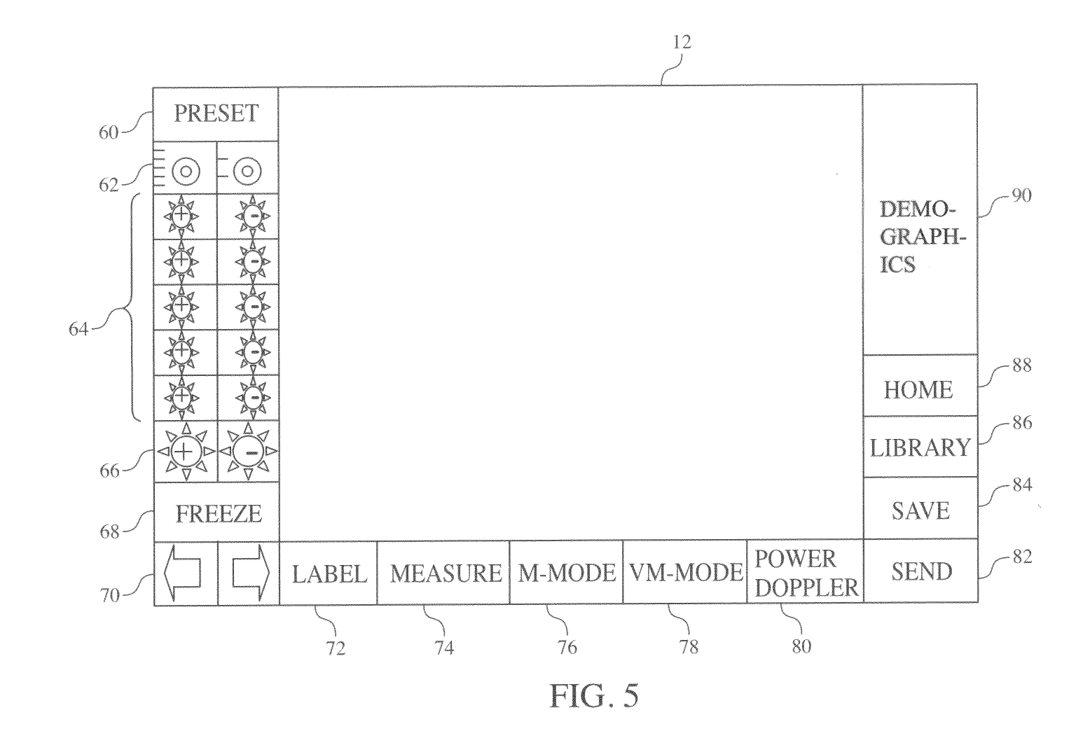

FIG. 5 illustrates various embodiments of the user interface 12. For such embodiments, the user interface 12 is embodied as a touch screen user interface on the display 18. The touch screen may utilize any suitable type of touch screen technology. For example, according to various embodiments, the touch screen may be a resistive touch screen, a surface acoustic wave touch screen, a capacitive touch screen, an infrared touch screen, etc. The touch screen may be utilized with any of the above-described ultrasound devices. However, for purposes of simplicity, the touch screen will be described in the context of its use with the ultrasound device 10.

As shown in FIG. 5, the touch screen includes a plurality of buttons which may be utilized to set and/or control various parameters of the ultrasound application. In general, the buttons are arranged in a logical order which tracts the sequence typically employed in an ultrasound application. For example, for a user who holds the transducer 20 in the right hand, the logical order of the buttons begins in the upper left hand corner of the display 18 and proceeds sequentially in a counterclockwise direction. The user may first press the preset button 60 to select a particular target (e.g., heart, abdomen, vascular, etc.). The selection of a particular target serves to invoke a corresponding algorithm which automatically sets the focus of the transducer 20 to a ballpark area/depth. The pressing of the preset button 60 may further invoke one or more image optimizing signal processing programs to enable image acquisition with a minimum of manual adjustment.

After placing the transducer 20 on the patient, the user may then press a first one of the depth buttons 62 to increase the depth (reduce the size of the image) or a second one of the depth buttons 62 to decrease the depth (increase the size of the image). The depth buttons 62 may also be utilized to center an area of focus to the middle of the display 18. One or more of the time gain compensation buttons 64 may then be pressed to lighten portions of the image associated with deeper signals or to darken the portions of the image associated with shallower signals. Similarly, the user may press a first one of the overall gain buttons 66 to make the entire image brighter or a second one of the overall gain buttons 66 to make the entire image darker.

Once the image is in the desired condition, the freeze button 68 may be selected to capture a static copy of the image at that point in time. If the user wishes to capture a static copy of the image at an earlier point in time, the user may select a first one of the time adjustment arrows 70 (e.g., the left facing arrow). The time increments associated with the left facing arrow may be predefined such that each press of the left facing arrow moves the image back one frame, one second, etc. Similarly, if the user wishes to capture a static copy of the image at a later point in time, the user may select a second one of the time adjustment arrows 70 (e.g., the right facing arrow). The time increments associated with the right facing arrow may be predefined such that each press of the right facing arrow moves the image back one frame, one second, etc.

If the user wishes to label something on one of the captured images, the user may press the label button 72, utilize an input device (e.g., a mouse, a trackball, etc.) to move a cursor over an area of interest then activate the device to open a text box, then utilize the alpha-numeric keys of the keyboard to enter the desired label. If the user wishes to measure something on one of the captured images, the user may press the measure button 74, utilize an input device to move a cursor over a first part of an area of interest, left click the device, utilize the input device to move the cursor over a second part of the area of interest, then right click the input device to determine a distance between the first and second parts of the area of interest.

In addition to working with static images, the user may utilize one or more of the plurality of buttons to work in real-time. For example, the user may press the motion mode button 76 to measure motion in the typical selected unidimensional linear front to back sample of the image. According to other embodiments, an anatomical m-mode button may be pressed to allow for unidimensional selection in an orientation other than the typical front-to-back m-mode. As shown in FIG. 5, the touch screen may also include a virtual mode button 78 which can be selected by a user. The power doppler button 80 may be utilized to doppler shift within a selected area of the image.

When the user desires to transmit a particular image from the ultrasound device 10 to another location, the user may press the send button 82. The sent image may be a static image, a full motion image, a clip of a full motion image, etc. In order to save a particular image to memory, the user may press the save button 84. The image may be saved to any suitable memory device such as, for example, an internal memory, an external hard drive, a flash drive, etc. According to various embodiments, the image may be saved to a flash drive which is integral with a removable transducer. If the user desires to access other images (e.g., for purposes of comparison to a particular captured image) for viewing on the display 18, the user may press the library button 86 to access and retrieve such other images.

For embodiments where the ultrasound device is in communication with a remote computing system, the user may press the home button 88 to exit from the ultrasound application and return to a different application available on the remote computing system. For embodiments where the user wishes to enter and save demographic information associated with the patient, the user may press the demographics button 90 to access one or more templates or text boxes, then utilize the alpha-numeric keys of the keyboard to enter the information.

Although the buttons shown in FIG. 5 are logically organized for a user who holds the transducer 20 in the right hand, it will be appreciated that according to other embodiments, the buttons are flipped so that the buttons are logically organized for a user who holds the transducer 20 in the left hand. For such embodiments, the preset button 60 would be in the upper right hand corner of the display 18, and the buttons would proceed sequentially in a clockwise direction. According to various embodiments, a user can select the logical arrangement of the buttons by pressing a left hand button (not shown) or a right hand button (not shown). Although only certain buttons are shown in FIG. 5, it will be appreciated that the touch screen may include any number of additional buttons which are typically utilized to manipulate, associate information with, and/or process an image.

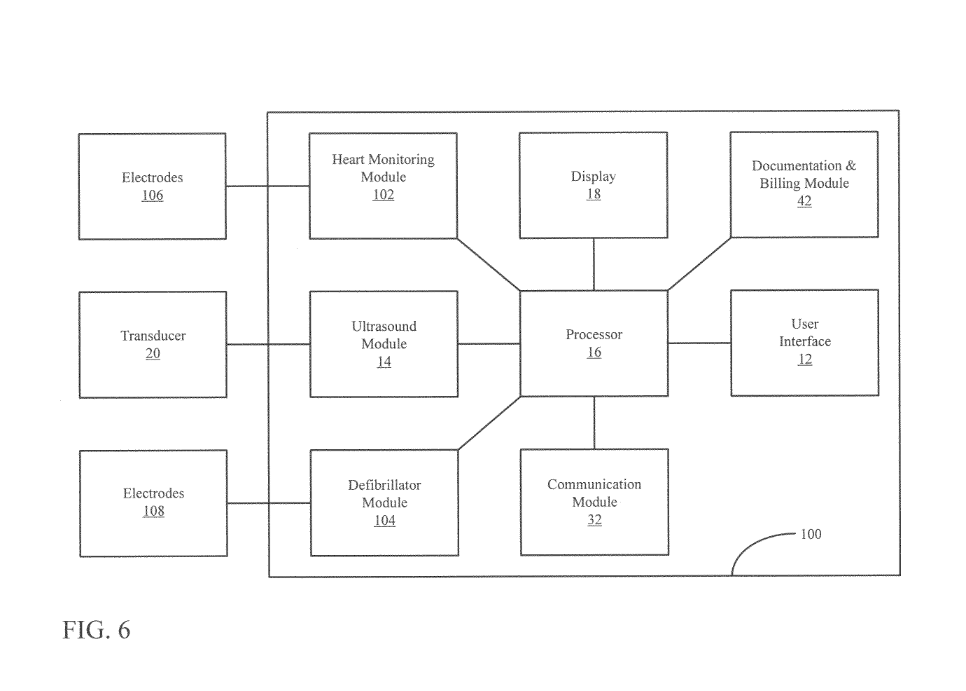

FIG. 6 illustrates a high level representation of an ultrasound device 100 according to various embodiments. The ultrasound device 100 may be similar to any of the ultrasound devices described herein before, but is different in that the ultrasound device 100 also includes a heart monitor module 102 in communication with the processor 16, and/or a defibrillator module 104 in communication with the processor 16. According to various embodiments, the heart monitor module 102 is embodied as a chip set similar to those, currently offered by, for example, Zoll Medical Corporation of Chelmsford, Mass., Philips, and/or Physio-Control of Redmond, Wash. The heart monitor module 102 is configured for digitizing signals received from any of a plurality of physiological sensors. The defibrillator module 104 may be embodied as a chip set similar to those offered by the above-referenced companies, and is configured for applying an appropriate waveform to electrically stimulate a patient's heart. According to other embodiments, the functionality of the ultrasound module 14, the heart monitor module 102, and the defibrillator module 104 may be integrated within a single chip set.

As shown in FIG. 6, one or more pairs of electrodes 106 may be communicably connected to the heart monitor module 102. Additionally, one or more pairs of electrodes 108 may be communicably connected to the defibrillator module 104.

The device 100 may be utilized to measure a wide variety of variables including at least one or more of the following: heart rate, electrocardiogram, pulse oximetry, invasive and non-invasive blood pressure measures, capnography, and body temperature. The device 100 may also be utilized to evaluate the volume of internal anatomical structures to assess physiological measures. For example, the volume of the heart, and thus the relative blood volume, of a patient may be easily assessed by the device 100. The device 100 may also be utilized to assess cardiac function through an electrocardiogram and address any arrhytmias through delivering an electric shock to the heart.

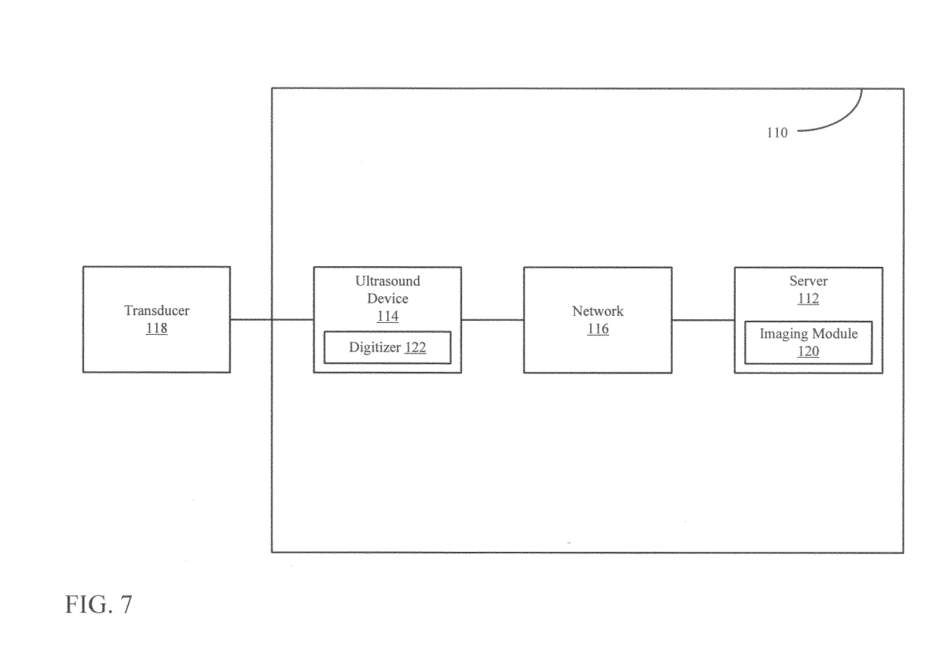

FIG. 7 illustrates various embodiments of a system 110. The system 110 includes a server 112, and an ultrasound device 114 communicably connected to the server 112 via a network 116. As shown in FIG. 7, a transducer 118 may be communicatively connected to the ultrasound device 114. Although only one ultrasound device 114 is shown in FIG. 7, it will be appreciated that the system 110 may include any number of ultrasound devices 114 communicably connected to the server 112. Additionally, although only one server 112 is shown in FIG. 7, it will be appreciated that the system 110 may include any number of servers 112.

The server 112 includes an imaging module 120 configured for generating an image representative of information captured by the transducer 118. The imaging module 120 may be implemented in either hardware, firmware, software or combinations thereof. For embodiments utilizing software, the software may utilize any suitable computer language (e.g., C, C++, Java, JavaScript, Visual Basic, VBScript, Delphi) and may be embodied permanently or temporarily in any type of machine, component, physical or virtual equipment, storage medium, or propagated signal capable of delivering instructions to a device. The imaging module 120 (e.g., software application, computer program) may be stored on computer-readable mediums such that when the mediums are read, the functions described herein are performed. For embodiments where the system 110 includes more than one server 112, the imaging module 120 may be distributed across a plurality of servers 112.

The ultrasound device 114 may be similar to any of the ultrasound devices described hereinabove. Thus, for such embodiments, a separate ultrasound module may be incorporated into each bedside ultrasound device. According to various embodiments, the ultrasound device 114 may be embodied as a smart monitor that includes a digitizer (e.g., an analog-to-digital converter) for digitizing the signal received from the transducer 118. After the signal is digitized, the ultrasound device 114 may then send the signal to the server 112 for processing. For such embodiments, instead of including a plurality of complete ultrasound modules (e.g., one at each bedside), a single ultrasound module is incorporated into the server 112, and the system 110 may simply include a smart monitor 114 at each bedside, wherein each of the smart monitors 114 are communicably connected to the server 112 via the network 116.

In general, the ultrasound device 114 and the server 112 each include hardware and/or software components for communicating with the network 116 and with each other. The ultrasound device 114 and the server 112 may be structured and arranged to communicate through the network 116 via wired and/or wireless pathways using various communication protocols (e.g., HTTP, TCP/IP, UDP, WAP, WiFi, Bluetooth) and/or to operate within or in concert with one or more other communications systems.

The network 116 may include any type of delivery system including, but not limited to, a local area network (e.g., Ethernet), a wide area network (e.g. the Internet and/or World Wide Web), a telephone network (e.g., analog, digital, wired, wireless, PSTN, ISDN, GSM, GPRS, and/or XDSL), a packet-switched network, a radio network, a television network, a cable network, a satellite network, and/or any other wired or wireless communications network configured to carry data. The network 116 may include elements, such as, for example, intermediate nodes, proxy servers, routers, switches, and adapters configured to direct and/or deliver data.

In operation, the ultrasound capabilities of the system 110 may be actuated at the ultrasound device 114 in any suitable manner. For example, according to various embodiments, the ultrasound capabilities may be actuated by an automatic logon of the transducer 118. Once the ultrasound capabilities are actuated, the information received by the ultrasound device 114 via the transducer 118 is digitized then forwarded to the server 112 via the network 116. At the server 112, the imaging module 120 processes the received information, generates an image representative of the information, and transmits the image to the ultrasound device 114 via the network 116 for viewing on the display 18 of the ultrasound device 114. By processing the information and generating the image at the server 112 in lieu of the respective ultrasound devices 114, the complexity and cost of each ultrasound device 114 is lower than each of the other ultrasound devices described hereinbefore, thereby decreasing the cost of the system 110.

According to various embodiments, the transducer 118 may be embodied as a pocket-sized personal transducer similar to the one described hereinabove. For such embodiments, the memory device removably connected to the transducer 118 may store a user identification and/or other user characteristics, and may announce itself to the system 110 once it is connected to a smart monitor 114 at the bedside of a patient. A more detailed description of such a pocket-sized personal transducer is provided hereinbelow with respect to FIG. 8.

FIG. 8 illustrates various embodiments of a transducer 130. The transducer 130 may be utilized with the system 110 of FIG. 7. The transducer 130 includes a cable 132 which has a first end 134 configured for connection to the ultrasound device 114 of the system 110, and a second end 136 configured for receiving any of a plurality of different detachable probes 138. The different detachable probes 138 may be embodied as, for example, a cardiology probe, an abdominal probe, an obstetrical probe, a vascular probe, etc.

According to various embodiments, at least one of the detachable probes 138 may include a thumb drive 140 which may be utilized to store the information received by the probe 138 of the transducer 130, and/or to store one or more of the images generated by the server 112. According to various embodiments, the system 110 automatically associates a given detachable probe 138 with a particular person (e.g., a physician) each time the detachable probe 138 is communicatively connected to the ultrasound device 114. According to other embodiments, the flash drive may also be accessed independently of the probe to download information to, for example, a desktop computer, a laptop, a server, etc.

According to various embodiments, the cable 132 is a dual function cable. One part of the cable is embodied as a micro-coaxial cable and is utilized to transmit image signals. A second part of the cable is embodied as a universal serial bus which allows for portable transducer access at the transducer, thereby eliminating the need to carry around a transducer which includes several feet of cable. According to various embodiments, the personal transducer is configured to recognize how many pins and which pins to utilize automatically. Additionally, according to various embodiments, the transducer 130 is configured such that the cable 132 is detachable at the probe/transducer end instead of at the ultrasound device end.

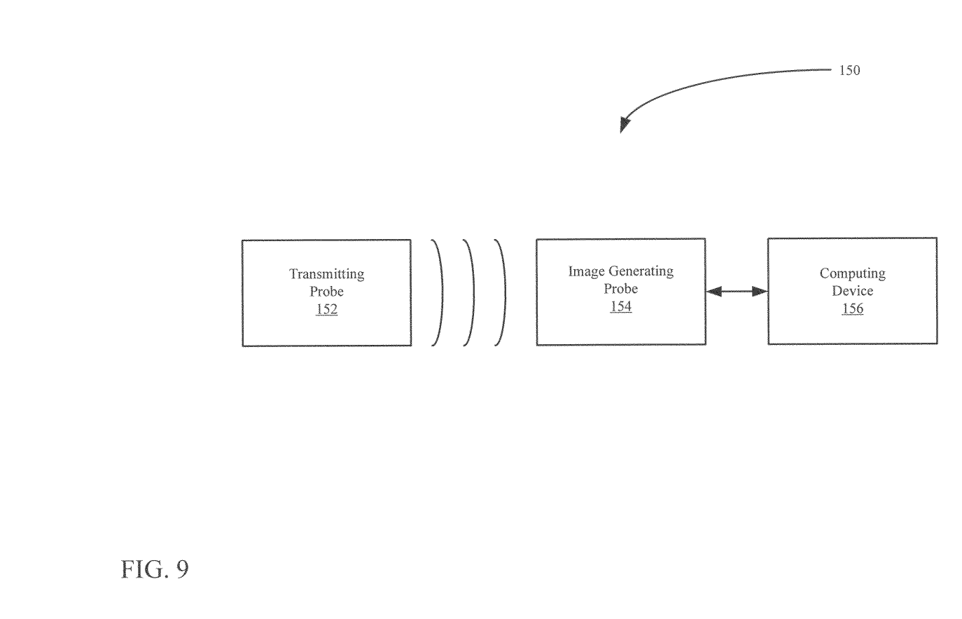

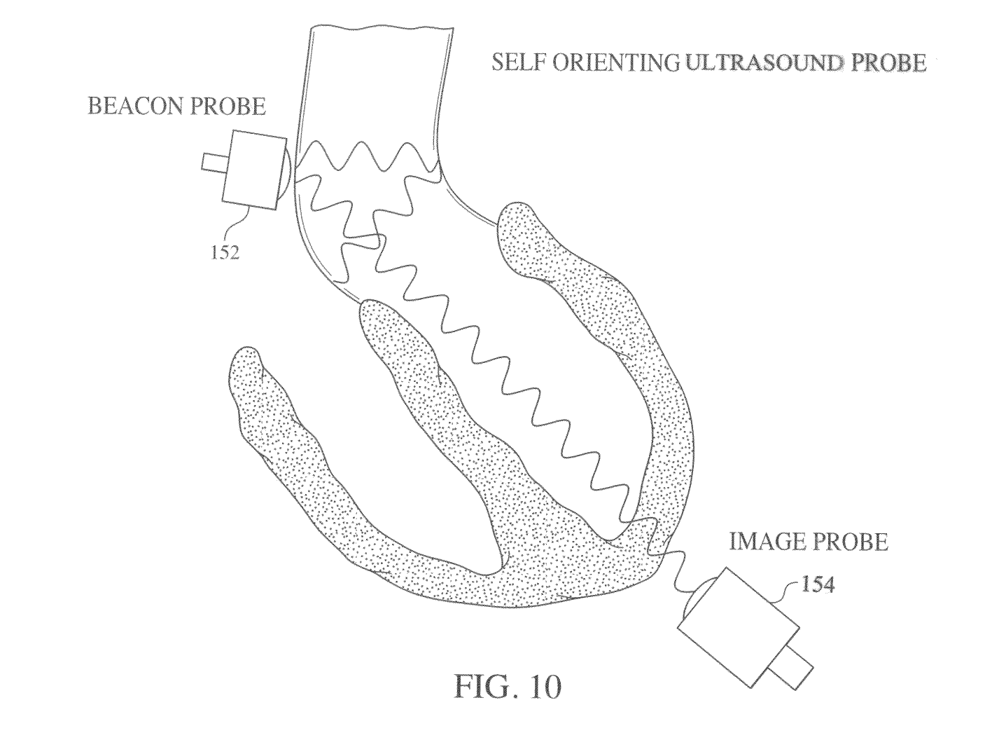

FIG. 9 illustrates a high level representation of an ultrasound system 150 according to various embodiments. As explained in more detail hereinbelow, the system 150 may be utilized for continuous ultrasonographic monitoring. For purposes of simplicity, the system 150 will be described in the context of continuous ultrasonographic montitoring of a heart. However, it will be appreciated that the system 150 may be utilized with structures other than a heart. The system 150 includes a first probe 152, a second probe 154, and a computing device 156 communicably connected to the second probe 154. The first probe 152 may be referred to as a transmitting probe or a beacon probe, and the second probe 154 may be referred to as an image generating probe. The computing device 156 is configured to analyze signals received from the second probe 154. An illustration of the placement of the first and second probes 152, 154 relative to a heart is shown in FIG. 10.

In general, cardiac ultrasound or echocardiography requires technically more difficult probe positioning than other ultrasound applications. Continuous monitoring, particularly important in any cardiac monitor device application, is essentially impractical for currently available probe configurations to be affixed in place in the exact position on a patient to provide benefits of continuous monitoring provides. Attempting to obtain views more ideal for gathering information best acquired by subtle probe repositioning and then reaffixing probe position are even more impractical. The system 150 may be utilized to realize continuous ultrasonographic monitoring which provides direct real time monitoring of actual cardiac activity and function rather than inferential information such as that obtained by monitoring electrical activity or even blood pressure. The information obtained via the continuous ultrasonographic monitoring may be obtained and trended realtime by a less skilled provider than a trained echocardiography technologist and in continuous form rather than the episodic viewing constrained by current echo technology. and echocardiography machine availability.

As explained hereinabove, the system 150 may be utilized to realize continuous ultrasonographic monitoring of the heart. By placing the transmitting probe 152 (the “beacon probe”) over the aortic position as shown in FIG. 10, the location at the upper right sternal border is used to preferentially auscultate aortic valve sounds or potentially other anatomic landmarks over large arteries with the image generating probe 154 affixed to the patients chest over the apical position, where the patients heartbeat is typically best palpated. A “beacon” signal, uniquely recognizable by virtue of unique frequency, pulse repetition, a combination of frequency and pulse repetition, by other digital signature, may be directed towards the aortic valve. The wavefront with the fewest internal reflections and the one essentially traveling directly down the aortic outflow tract without internal cardiac reflection will strike the image creating crystals of the image generating probe 154 first and in a sequence from which the image generating probe 154 would generate a signal which is analyzed by the computing device 156 to determine the exact vector of the long axis of the left ventricle extending through the aortic outflow tract. By determining this position, beam forming elements within the image generating probe 154 are activated in a manner which directs the image forming beam up the axis of the aortic outflow tract, thereby creating a typically desired echocardiographic view of the heart. By virtue of knowing this axis, other desired views of the heart obtainable from the apical poison can be deduced from the aortic outflow axis. With a few simple ultrasonographic measurements, other views obtainable from the apex may be automatically calculated by the computing device 156 and then procured automatically at the desire of the clinician. The axis may be automatically and continually recalibrated by keeping the beacon probes 152 affixed to the chest and thereby maintaining proper image beam orientation to facilitate continuous capture and comparable images over time. A manual recalibration may also be triggered at any time by manually triggering a beacon “pulse” or reapplying the beacon probe 152 and triggering a pulse.

According to various embodiments, the patient interface for both the beacon probe 152 and the image generating probe 154 are oriented 90° to the axis of the respective probe to allow for a simple fixation to the chest wall for continuous monitoring. Although the system 150 has been described in the context of a cardiac application, it will be appreciated that the system 150 may also be utilized in noncardiac applications where automatic positioning using a vascular beacon signal signature could be used to direct image generating probe beam forming elements to view other anatomic structures automatically and continually such as freshly transplanted organs, vascular surgical repairs, etc.

Nothing in the above description is meant to limit the invention to any specific materials, geometry, or orientation of elements. Many part/orientation substitutions are contemplated within the scope of the invention and will be apparent to those skilled in the art. The embodiments described herein were presented by way of example only and should not be used to limit the scope of the invention.

Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

What is claimed is:

1. A portable ultrasound device, comprising:
a shock and vibration resistant housing; and
an ultrasound module positioned within the housing, wherein the ultrasound module is configured for:
transmitting control signals to a transducer; and
digitizing echo signals received from the transducer;
a processor positioned within the housing, wherein the processor is communicably connected to the ultrasound module, and wherein the processor is configured to generate an image based on the digitized echo signals; and
a display communicably connected to the processor.

2. The portable ultrasound device of claim 1, wherein the housing comprises magnesium.

3. The portable ultrasound device of claim I, further comprising a user interface communicably connected to the processor.

4. The portable ultrasound device of claim 3, wherein the user interface is a touch screen.

5. The portable ultrasound device of claim 4, wherein the touch screen includes a logical ordering of buttons.

6. The portable ultrasound device of claim 5, wherein the touch screen includes a button for changing the logical ordering from a right hand logical ordering to a left hand logical ordering.

7. The portable ultrasound device of claim 1, further comprising a communication module communicably connected to the processor, wherein the communication module is configured for wirelessly transmitting information to a remote computing system.

8. The portable ultrasound device of claim 1, further comprising a documentation module communicably connected to the processor, wherein the documentation module is configured for appending patient information to an image.

9. The portable ultrasound device of claim 1, further comprising a billing module communicably connected to the processor, wherein the billing module is configured for associating billing information with an image.

10. A portable device, comprising:
an ultrasound module, wherein the ultrasound module is configured for:
transmitting control signals to a transducer; and
digitizing echo signals received from the transducer;
a processor communicably connected to the ultrasound module, and wherein the processor is configured to generate an image based on the digitized echo signals;
a display communicably connected to the processor; and
at least one of the following communicably connected to the processor:
a heart monitor module; and
a defibrillator module.

11. The portable device of claim 10, further comprising a user interface communicably connected to the processor.

12. The portable device of claim I 1, wherein the user interface is a touch screen.

13. The portable device of claim 12, wherein the touch screen includes a logical ordering of buttons.

14. The portable device of claim 13, wherein the touch screen includes a button for changing the logical ordering from a right hand logical ordering to a left hand logical ordering.

15. The portable device of claim 10, wherein the device further comprises a shock and vibration resistant housing.

16. A system, comprising:
a device configured to digitize a signal received from a transducer; and
a server communicably connected to the device, wherein the server is configured to generate an image based on the digitized signal.

17. The system of claim 16, wherein the system further comprises a plurality of devices communicably connected to the server.

18. The system of claim 16, wherein the device comprises a monitor.
